(19) United States
(12) Patent Application Publication
(10) Pub. No.: US 2008/0213823 A1
(43) Pub. Date: Sep. 4, 2008

(54) CAPILLARY-CHANNELED POLYMER FILM FLOW CYTOMETRY

(76) Inventors: Kenneth A. Christensen, Clemson, SC (US); R. Kenneth Marcus, Seneca, SC (US)

Correspondence Address: NEEDLE & ROSENBERG, P.C., SUITE 1000, 999 PEACHTREE STREET, ATLANTA, GA, 30309-3915, US

(21) Appl. No.: 12/036,894

(22) Filed: Feb. 25, 2008

Related U.S. Application Data
(60) Provisional application No. 60/891,363, filed on Feb. 23, 2007.

(51) Int. Cl. C12Q 1/06; C12M 1/00

(52) U.S. Cl. 435/39; 435/287.1; 435/288.7

(57) ABSTRACT

The present invention relates to methods for counting, sorting, and manipulation of cells, organelles, and/or cellular material using capillary-channeled polymer films.

### PRIORITY

This application claims the benefit of Provisional Application Ser. No. 60/891,363 filed on Feb. 23, 2007, and the entire disclosure of Provisional Application Ser. No. 60/891,363 is incorporated herein by reference in its entirety.

### FIELD OF THE DISCLOSURE

The present invention relates to methods for counting, sorting, and manipulation of cells, organelles, and/or cellular material and/or debris using capillary-channeled polymer films.

### BACKGROUND

Rapid and inexpensive counting of cells is an important aspect of biochemistry. Cell types have different surface protein, carbohydrate, and lipid markers. As such, these differences can be utilized to obtain differential cell manipulation. Flow cytometry and Fluorescent activated cell sorting (FACS) are techniques currently utilized to count, separate, and to identify cell types. An additional existing technique is a bead-based extraction or immunoprecipitation or “pull-down”, which involves multiple steps to sufficiently isolate and concentrate the targeted cells into a final enriched fraction ready for counting, analysis, or further manipulation.

As disclosed in U.S. patent application Ser. No. 11/546,602 published as U.S. 2007/0071649, incorporated herein by reference in its entirety, capillary-channeled polymer fibers can be used as the stationary phase media for spectroscopic analysis of chemical species. This disclosed technology is a suitable replacement for reversed-phase chromatography affording a rapid and inexpensive method for separating chemical species that is adaptable to all manner of analytical analysis, inter alia, UV/visible spectroscopy, fluorescence spectroscopy, and infrared spectroscopy.

A need exists for a method to count, separate and detect cells in an inexpensive and rapid manner that allows for on-time analysis. Applications of such methods would include cell counting without the need to concentrate and/or further isolate the cells or cellular matter being analyzed.

### SUMMARY OF THE INVENTION

Disclosed herein are articles of manufacture and systems that provide a rapid, inexpensive, and reproducible method for analyzing and counting cells and/or other cellular matter. Little or no sample preparation is necessary when utilizing these methods.

Disclosed are methods for detecting and/or counting cells, cellular organelles or cellular material/debris, comprising:
a) providing a fluid conduit having a first end and a second end disposed opposite the first end, the conduit having at least one polymer film disposed therein, with the at least one polymer film defining a plurality of capillary channels capable of wicking a fluid;
b) defining a detection probing window position at a location along the conduit between the first end and the second end of the conduit
c) aligning an instrument aligned with the probing detection window position, with the instrument being configured for detecting and/or counting cells, or cellular material;
d) moving movement of fluid containing the cells or cellular material through the capillaries; and
e) detecting of the number of cells and/or cellular materials with the instrument.

### BRIEF DESCRIPTION OF THE DRAWINGS

FIG. 1 depicts a cross-sectional representation of a cell comprising or cellular material comprising liquid flowing through channeled fibers placed in a single column formed by a 0.25-inch (about 4.5 mm) diameter tube, including an expanded view window showing the end-on shape of the fibers and the potential irregular packing of the fibers in the column.

FIG. 2A is a schematic representation of a separation apparatus in accordance with the present invention.

FIG. 2B is a schematic representation of a spectroscopic detector aligned with a probing position on the column of FIG. 2A.

FIG. 3 is a schematic representation of a capillary electrophoresis system in accordance with the present invention.

FIG. 4 is a perspective view of a lab on a chip capable of carrying a single surface-channeled fiber in accordance with the present invention.

FIG. 5 schematically represents the use of a fiber/film assembly used to sample an analyte-containing liquid for subsequent transport to a separate instrument having an appropriate detector for analysis of immobilized solutes in accordance with the present invention.

FIG. 6 depicts a capillary-channel having a plurality of surface channel modifications.

FIG. 7 depicts the use of capillary-channeled films in a lateral flow assay.

FIG. 8A depicts a single image of labeled TH-1 cells obtained by a time-lapse movie of the cell count of Example 1.

FIG. 8B depicts a histogram of number of cells vs. intensity from the time-lapse movie recorded during Example 1.

FIG. 9 depicts the flow Cytometry histogram using a Becton-Dickenson FACScan flow cytometer (488 nm excitation, 535 nm fluorescence emission) of cell counts from Example 1.

FIG. 10 depicts a transmitted light (bright field) image of a capillary-channeled film where a proteinaceous recognition substrate/element diluted in phosphate buffer +50% glycerol was printed within a single channel.

### DETAILED DESCRIPTION

Throughout the description and claims of this specification, the word “comprise” and variations of the word, such as “comprising” and “comprises,” means “including but not limited to,” and is not intended to exclude, for example, other additives, components, integers or steps.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A weight percent of a component, unless specifically stated to the contrary, is based on the total weight of the formulation or composition in which the component is included.

By “contacting” is meant the physical contact of at least one substance to another substance.

By “sufficient amount” and “sufficient time” means an amount and time needed to achieve the desired result or results, e.g., dissolve a portion of the polymer.

By “cell” is meant a cell that is living or fixed or that has been stained or otherwise chemically or biologically marked or otherwise differentiated from one or more other cells, or which is chemically or biologically marked or otherwise differentiated by a means for selectively interacting with the cell.

By “organelle” is meant a cellular substructure, for example, mitochondria.

By “cellular material” is meant any macromolecules, fragments of macromolecules, or individual organic compounds or other cellular debris that can be found in a cell, for example, fragments of cell membrane, enzymes, nucleotides, amino acids, peptides, proteins, oligosaccharides, polysaccharides, and the like.

By “reservoir” is meant a location within a channel that can serve as the point at which a sample or an analyte is added, for example, a “receiving reservoir” or “reservoir for receiving.” Additional reservoirs include “reagent reservoir” or “reservoir for one or more reagents” which is a reservoir that contains one or more reagents that can react, change, or otherwise modify one or more of the substances that comprise the sample or analyte.

“Polymer” as used herein refers to any type of polymer including, for example, a homopolymer, a copolymer, a block copolymer, a random copolymer, and the like.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is to be understood that the articles of manufacture and methods are not limited to specific embodiments, specific analytical techniques, or to particular reagents unless otherwise specified, and, as such, may vary. It is also to be understood that the terminology used herein is for the purpose of describing particular embodiments only and, as such, is not intended to be limiting.

Capillary-channeled polymer (C-CP) films possess unique physical and chemical properties that allow for the manipulation of cells and cellular material (both animal and plant) within the channel structure in a massively parallel fashion. The channel diameters can be controlled such that individual cells can be placed within them and manipulated (moved) through the use of capillary forces or microfluidic pumping, The channels can be used to isolate cells from one another such that they can be counted, sorted, and characterized by various means. Channels having different diameters allow for sorting and counting based on size. In this case, cells can be sorted, counted and monitored by measuring the velocity of cells moving through the channels. A variety of surface chemistries can be affected on the polymer surfaces to allow attachment of small molecules, peptides, proteins, DNA/RNA, antibodies, or other molecules with biological function, for example, having the ability to recognize specific domains or receptors on a cell. These derivatized channels can be used to separate, sort and count cells based on specific interactions with these surfaces. These attached ligands can also be implemented in a massively parallel fashion to allow an entire library of ligands to be individually localized within single channels of these films. Cells bound to these channels can be further examined to study cellular morphology, signaling, chemotaxis, and other cellular processes in response to different external stimuli. There are no commercially available systems that provide massively parallel processing of cells in the manner disclosed herein.

Further disclosed herein are methods for detecting and/or counting cells, organelles, or cellular material wherein the conduit having at least one polymer film deposed thereon comprises a polymer that is modified to interact with the cells and/or cellular material.

In another disclosed embodiment, the methods are directed to the use of a device for visually detecting and determining the number of at least one cellular species in a fluid, which includes at least one polymer film, the at least one polymer film defining a plurality of capillary channels capable of wicking a fluid, and at least one means for selecting one cellular type over other cellular types present, wherein the method utilizes an indicator disposed on the surface of the at least one polymer film, the cellular selective indicator exhibiting a visually distinguishable response in the presence of the at least one cellular species.

In a yet further disclosed embodiment, the methods are directed to the use of a device comprising at least one cellular selective indicator that can include at least a first cellular selective indicator and a second cellular selective indicator for detecting at least two differing cellular species. This embodiment includes an iteration wherein the first cellular selective indicator can be disposed on the surface of the at least one polymer film or modified polymer film at a desired interval from the disposition of the second cellular selective indicator. In another iteration, the first cellular selective indicator and the second cellular selective indicator can be proximal, adjacent, or juxtaposed in register to each other in separate channels and at a position approximate or co-equal to the position of the other indicator along the length of the at least one polymer channel or modified polymer channel.

In a still further disclosed embodiment, the methods are directed to detecting at least one cell species, cellular organelle and/or cellular material/debris in a fluid, which includes providing at least one polymer film or modified polymer film having a first end and a second end, the at least one polymer film defining a plurality of capillary channels capable of wicking a fluid, disposing on the surface of the at least one polymer film or modified polymer film at least one cellular selective indicator, wherein the cellular selective indicator can exhibit a visually distinguishable response in the presence of the at least one cellular species and/or cellular material, exposing the first end of the at least one polymer or modified polymer film to a fluid, and observing the at least one polymer or modified polymer film to determine the presence of a visually distinguishable response indicating the presence of the at least one cellular species and/or the presence of at least one cellular material. In one iteration, the visually distinguishable response exhibited by the cellular selective indicator comprises a visible color change.

In a yet still further disclosed embodiment, the methods are directed to detecting at least one cellular species, organelle, or cellular material in a fluid, which includes providing at least one polymer film or modified polymer film having a first end and a second end, the at least one polymer film defining a plurality of capillary channels capable of wicking a fluid, disposing on the surface of the at least one polymer film or modified polymer film at least one cellular selective indicator, wherein the cellular selective indicator can emit a response distinguishable by an apparatus or instrument that can detect absorbance, fluorescence, other luminescence, or scattered light (e.g. Raman scattering) in the presence of the at least one cellular species and/or cellular material, exposing the first end of the at least one polymer or modified polymer film to a fluid, and detecting with an apparatus or instrument the presence of a distinguishable spectroscopic response indicating the presence of the at least one cellular species and/or the presence of at least one cellular material.

### Polymeric Substrates

In general use, the present channeled polymer films (20) tend to have a very strong wicking action for a variety of liquids, including water. The separation and filtration capabilities of the present channeled polymer films are described in detail in the present parent, U.S. Ser. No. 10/485,701, filed Feb. 3, 2004, which is hereby incorporated by reference in its entirety.

The present disclosure relates to polymeric substrates having one or more fluid conduits disposed thereon. The conduits comprise polymeric film(s) and or fiber(s) that comprise one or more capillary channels. The capillary channels direct a liquid sample containing either cells, organelles, cellular material/debris, or mixtures thereof, from the point at which the sample is applied to the channel to a point wherein an interaction with the sample takes place. The sample is moved along the channels by fluid wicking. A channel is said to be “active” if it comprises at least one means for analyzing or detecting a substance. A channel is said to be “passive” if it does not comprise at least a means for analyzing or detecting a substance. In one embodiment, active channels comprising a means for detecting a substance are separated by one or more passive channels. The passive channels are not aligned with a means for detecting substances contained in the sample fluid. In addition, a dye or other signal to the user, such as a letter, number, chemical or biological symbol, can be added to the channel, such as in staining or embossing a portion of the channel to signal to the user that a channel is active or passive. In the case of active channels having a demarcation, the symbol can be activated by the presence of the solvent or other additive used make up the sample. The signal or demarcation can be permanent or temporary and serve to signal to the user the presence of a sample.

The capillary channels (either films or fibers) of the disclosed polymeric substrates can comprise any polymer that can be extruded. In one embodiment the polymer can be extruded at a temperatures of from about 150° C. to about 350° C. Thermoplastics are excluded from the polymers that can form the polymeric substrates. As such, the polymers that comprise the substrates can be any polymer that promotes or otherwise provides wicking of the analyte along the channel from the receiving reservoir to the detecting means.

Non-limiting examples of polymers includes poly(ethylene terephthalate) having the formula:

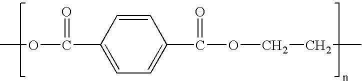

wherein the index n has a value such that the poly(ethylene terephthalate) has an average molecular weight of from about 5000 Da to about 500,000 Da; nylon, for example, Nylon 6,6 having the formula:

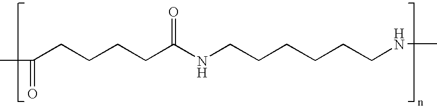

wherein the index n has a value such that the nylon has a weight average molecular weight of from about 5000 Da to about 500,000 Da; and polypropylene having the formula:

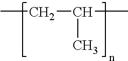

wherein the index n has a value such that the polypropylene has a weight average molecular weight of from about 5000 Da to about 500,000 Da.

In one embodiment, the polymer has a molecular weight of from about 5000 Da to about 120,000 Da. In another embodiment, the polymer has a molecular weight of from about 5000 Da to about 50,000 Da. In a further embodiment, the polymer has a molecular weight of from about 10,000 Da to about 30,000 Da. In yet further embodiment, the polymer has a molecular weight of from about 20,000 Da to about 50,000 Da. In a still further embodiment, the polymer has a molecular weight of from about 5000 Da to about 12,000 Da. In yet still further embodiment, the polymer has a molecular weight of from about 8000 Da to about 15,000 Da.

One measure of a characteristic of the polymer(s) suitable for use in forming the disclosed substrates is the intrinsic viscosity (I.V.) that is measured in deciliter per gram (dl/g) and is dependent upon the length of its polymer chains. For embodiments comprising polymer having a higher I.V., the polymers will have a longer chain length. For embodiments that comprise films or film coatings applied to a fibrous base, the intrinsic viscosity is about 0.6 dl/g. For extruded substrates, for example, extrusions of poly(ethylene terephthalate) the intrinsic viscosity is from about 0.62 dl/g to about 0.9 dl/g.

One aspect of the disclosed methods relates to fluid conduits comprising a capillary-channel polymer that is surface modified. The surfaces of the polymers can be treated with one or more reagents that provide a reactive moiety that can serve as a point of chemical attachment or for hydrostatic attachment of a marker, a molecular recognition element, or a reagent. For example, poly(ethylene terephthalate) can be treated with base, for example, sodium hydroxide, to provide a plurality of carboxy and hydroxy units on the surface of the polymer for chemically attaching or hydrostatically attaching a reagent that can modify the substances as they pass over the reagent well, or can be used to detect or measure a property of the substances at the detection device. The modification can also change the flow rate at which the cells are wicked. In addition, the modifications can preferentially slow, abate, or modify the flow rate of other materials in the fluid, thereby allowing free passage of the cells across the detection device.

For example, poly(ethylene terephthalate) can treated with a base (for example, NaOH) to form a plurality of surface polymer chains having one or more active groups as depicted in the following scheme.

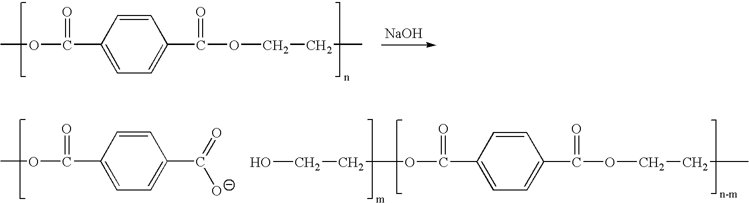

After forming a reactive group on the surface of the polymer, a substance or reagent can optionally be reacted with the active groups. This method can afford a means for cell surface recognition or can serve as a means for applying a first or second label or tag that is specific to the cells which are being counted. The following scheme depicts one example of attachment of a cell-recognizing adduct to the modified polymer.

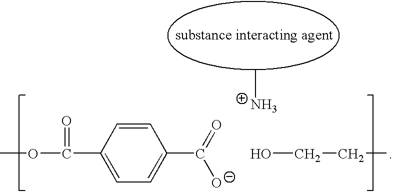

Aside from treatment with a base, the polymer surface can be modified by any method chosen by the formulator, for example, by treatment with ozone gas, a solution having ozone gas dissolved therein, or with any oxidant capable of providing a plurality of carboxyl groups on the polymer surface.

In a like manner, nylon can be treated with a base to afford carboxyl and amino groups for attachment of a substance-interacting agent. Polypropylene can be treated with ozone to afford a surface with carboxyl groups and other polar sights of attachment.

The disclosed methods relate to wicking a fluid within a fluid conduit that comprises one or more polymer fibers disposed thereon, wherein the polymer film can form one or more capillary channels that can have a width of from about 10 μm to about 100 μm. In addition, the channels can be capped at both ends or at one end.

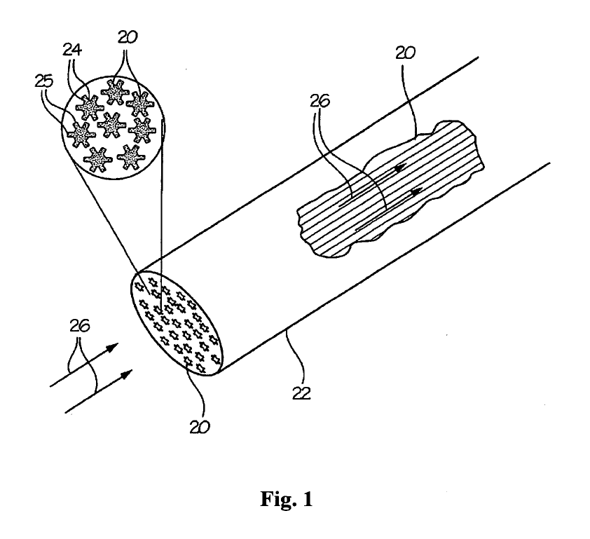

As shown in FIG. 1, bundles of capillary-channeled polymer fibers 20 are packed into a column 22 that is formed by a tube having a uniform circular inside diameter of 0.25 inches and a length of 12 inches. The dimensions of the column 22 can be any size that is adaptable to the present methods. In one embodiment, the length of each fiber 20 is substantially the same as the length of the column 22 and is disposed to extend within the column 22 over substantially the entire length of the column 22. In another embodiment, fibers 20 have lengths that are shorter than the length of the column 22. As such, various embodiments can include fibers of various lengths.

FIG. 1 also depicts an inset that illustrates one possible embodiment of the capillary-channel fibers. As shown schematically in cross-section in the expanded view of the inset, each fiber strand 20 has six co-linear channels 24 extending the entire length of the exterior surface of the fiber 20. Each channel 24 is defined by a pair of opposed walls 25 that extend generally and longitudinally and form part of the exterior surface of the fiber 20. As depicted, these channels 24 and walls 25 extend down the entire length of the fiber 20 parallel to the longitudinal axis of the fiber 20 and are, in one or more iterations, nominally co-linear on each fibers 20. The result is that in this embodiment the same co-linear channels 24 exist along the entire length of the column 22. This configuration of the capillary-channeled fibers illustrated in FIG. 1 is an example of the articles that can be used to count or sort cells and/or cellular materials. For instance, the number and/or cross-sectional shape of the channels can vary from that shown in the figures.

In another embodiment, the fibers 20 when packed into a bundle that lies along the entire length of the column 22, can have in one iteration one or more, or in another iteration all, of the fibers 20 in the bundle rotate about its/their own axis or the axis of the column 22 over the entire length of the column. This embodiment results in a column having a plurality of twisted capillary-channeled fibers 20 laying from one end of the column 22 to the opposite end.

In some of the disclosed embodiments, a device can be provided to deliver a cell-containing fluid or cellular-material-containing fluid to column 22 and thereby providing a means to continuously pass a sample-containing solution through the channels 24 of the fibers 20. The flow of liquid through the column 22 is schematically indicated by the arrows designated by the numeral 26 in FIG. 1. A portion of the column 22 is cut away in the view shown in FIG. 1 for the purpose of illustrating the flow of liquid 26 through the column 22 along the fibers 20 arranged with their longitudinal axes parallel to the longitudinal axis of the column 22. The diameter of each fiber 20 can be from about 10 to about 80 micrometers.

The sample-containing fluid is carried along the column by the capillary action provided by the polymers that comprise channels 24 of the films 20. In one embodiment, the fluid can move through the column with macro-level capillary action between the films in addition to or alternative to the micro-level capillary action through the channels 24 of the individual fibers 20.

The disclosed polymeric substrates can comprise any polymer that can be extruded and/or spin-melted. In one embodiment the polymer can be extruded at a temperature of from about 150° C. to about 350° C. Thermoplastics are excluded from the polymers that can form the polymeric substrates. As such, the polymers that comprise the substrates can be any polymer that promotes or otherwise provides wicking of the analyte along the channel from the receiving reservoir to the detecting means.

One aspect of the disclosed methods relates to an apparatus comprising surface-modified polymers. The surfaces of the polymers are treated with one or more reagents that provide a reactive moiety that can serve as a method for altering the flow rate of cells or cellular material carried by wicking of the fluid through the capillary channels. For example, poly(ethylene terephthalate) can be treated with base, for example, sodium hydroxide, to provide a plurality of carboxy and hydroxy units on the surface of the polymer for electrostatically or hydrostatically attaching the surface of cells or attracting one or more cellular materials. Modified polymer surfaces can assist in providing a slower or faster rate of cellular flow.

For example, poly(ethylene terephthalate) can be treated with a base (for example, NaOH) to form a plurality of modifications on the surface of the polymer by breaking a plurality of chemical bonds, for example, hydrolyzing an ester bond of a polyester as depicted in the following scheme.

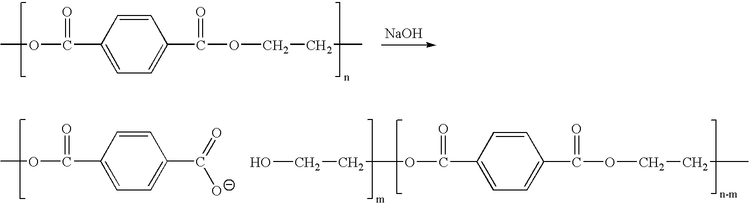

After forming a reactive group on the surface of the polymer, a substance or reagent that can react with one or more of the substances to be detected, can be chemically or electrostatically attached to the surface of the polymer at a reagent well, for example, as depicted herein below in the following scheme.

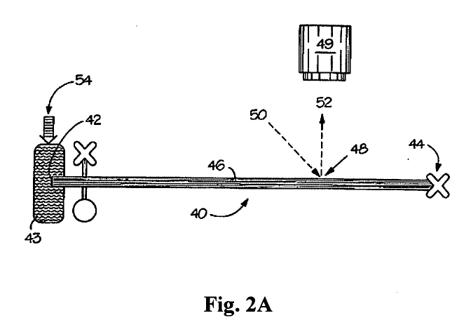

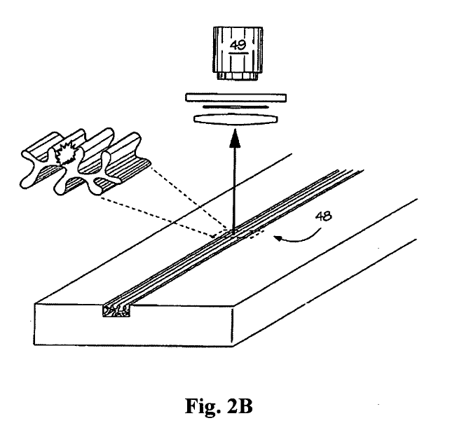

In general, the process used to make these channeled polymer fibers and films 20 is amenable to any polymer that can be spin-melted. For example, channeled fibers and films 20 may be melt-spun from any of a number of different polymer precursors. A non-limiting list of exemplary materials from which the fibers and films of the invention can be formed can include polypropylene precursors, polyester precursors, polyaniline precursors, precursors composed of polylactic acid, and nylon precursors. FIG. 2A schematically illustrates a simple separation apparatus containing a plurality of packed fibers, such as the capillary channeled fibers, as illustrated in FIG. 1. Specifically, fluid conduit 40 has a first end 42 associated with a fluid source 43 and a second, terminal end 44. Films 46 are disposed within the conduit, extending between the first end and the second end. A probing position 48 is defined on the conduit between the first and second ends. A spectroscopic detector 49, further illustrated in FIG. 2B, is aligned with the probing position and configured for detecting cells, organelles, and/or cellular material in a fluid moving through the conduit and along the length of the polymer film. FIG. 2A schematically represents an incident photon 50 and a scattered, transmitted or fluorescent photon 52 being monitored by the detector 49.

In one embodiment the fibers packed in conduit 40 are surface-channeled films but other cross-sectional geometries may also be employed, in accordance with the disclosed methods, as long as capillary channels are formed between the films. Channeled fibers and films provide for wicking ability on a single film basis.

A fluid containing cells, organelles, or cellular material/debris is moved through the capillaries defined by the polymer fibers and films to a detection device located at probing position 48 wherein the cells can be counted or the organelles and/or cellular material can be detected by a detection method such as IR absorbance, UV-VIS absorbance, fluorescence, Raman spectroscopy, mass spectrometry or any other suitable method.

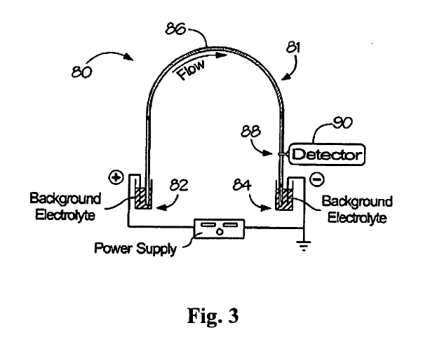

One method for moving the fluid through a fluid conduit in accordance with the disclosed methods is electro-osmosis. FIG. 3 schematically illustrates a capillary electrophoresis system 80 in which the capillary 81 is packed with fibers defining capillary channels. Capillary 81 is a fluid conduit having a first end 82 and a second end 84. Fibers 86 are disposed within the conduit, extending between the first end and the second end. A probing position 88 is defined on the conduit between the first and second ends. A spectroscopic detector 90 is aligned with the detection window and configured for detecting cells, organelles, and/or cellular material in a fluid moving through the conduit and along the length of the polymer fibers. In this system, the fluid migrates under the influence of an electrostatic potential, rather than relying merely on the wicking action of the fibers or an external pump.

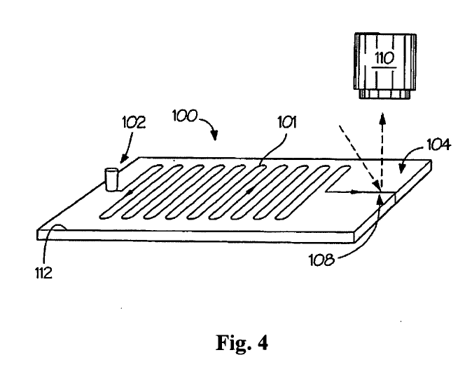

The two embodiments described above are directed to a separation apparatus having capillaries defined by a plurality of packed surface-channeled fibers. It should be noted that a single surface-channeled film as described herein may also be employed. One embodiment that relates to the use of single films in accordance with the present invention is as an extraction probe for solid-phase extraction. In a further embodiment, single surface-channeled fibers and films are particularly suited for use in microfluidic devices known as “lab on a chip” devices. As depicted in FIG. 4, microfluidic device 100 includes a single surface-channeled fiber or film 101, which, in this embodiment, defines a fluid conduit having a first end 102 and a second end 104. A probing position 108 is defined along the film between the first and second ends. A spectroscopic detector 110 is aligned with the detection window and configured for detecting cells, organelles, and/or cellular materials in a fluid moving along the length of the polymer fiber or film. The fiber or film is mounted within a channel of a polymeric, silicon, or glass chip 112 in a similar manner to that shown in FIG. 2B.

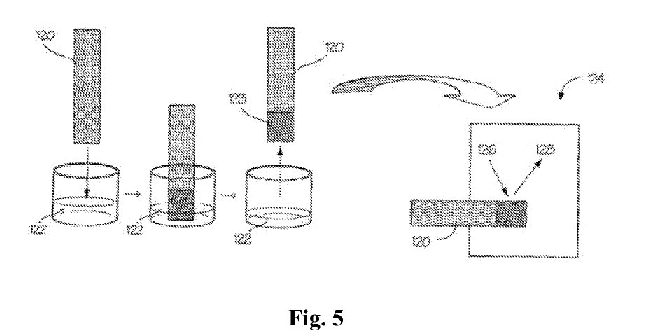

In other embodiments, the detector is aligned with a detection window on one or more films as described herein and the film is employed as a means to gather a fluid and take it to a detector. Thus, at least one polymer film as described herein is exposed to a fluid such that the fluid wicks onto the film and the at least one film is then positioned in an instrument configured for detecting cells, organelles, and/or cellular materials of interest. FIG. 5 illustrates a plurality of films (or, alternatively, a single channeled film) in the form of a test strip 120 being exposed to a test solution 122 such that at least some of the solution wicks onto the films as is shown at 123. The test strip is then transported to an instrument 124 for analysis using an appropriate probe beam and detector. For example, an incident photon 126 and a scattered, transmitted or fluorescent photon 128 are represented schematically. Alternatively, the probe beam may identify cellular material suitable for analysis by mass spectrometry.

All of the above-described systems are directed to the use of one or more films as a stationary phase for the transport of fluid, with a spectroscopic detector employed for detecting the presence of cells, organelles, and/or cellular material on the surface of the film or films. The disclosed methods further comprise systems in which, as above, one or more films act as a stationary phase for the transport of fluid, but a spectroscopic detector is not employed for detecting the presence of species. Rather, the presence of at least one cellular species, organelle, and/or cellular material is indicated by a visually detectable response or by use of a detector.

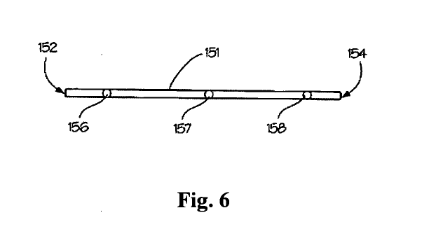

A film surface can be modified by the presence of at least one chemically selective indicator. In the presence of the investigated cells, organelles, and/or cellular material, the chemically selective indicator produces a visually distinguishable response, for example, a color change. In one embodiment of the disclosed methods, only one chemically selective indicator is present to test for the presence of one particular cell, organelle, and/or one type of cellular material. In another embodiment of the disclosed methods, two or more indicators are employed to test for the presence of at least two differing cells, organelles, and/or cellular materials. In a further aspect, the chemically selective indicator is such that it can detect the presence of more than one cell-type, organelle, and/or type of cellular material. FIG. 6 depicts an iteration of this embodiment. A single surface-channeled film 151 defines a fluid conduit having a first end 152 and a second end 154. The film surface has been modified to have a first material recognition substrate 156, a second material recognition substrate 157, and a third material recognition substrate 158. In this embodiment the three substrates are spaced from each other along the length of the film. Therefore, a fluid moves along the film from the first end 152 to the second end 154; the presence of a possible first cellular species is indicated by a visually distinguishable response of material recognition substrate 156, the presence of a possible second solute cellular species is indicated by a visually distinguishable response of the second material recognition substrate 157, and the presence of a possible third cellular species is indicated by a visually distinguishable response of the third material recognition substrate 158. The absence of any one of these species is confirmed by the absence of the respective response.

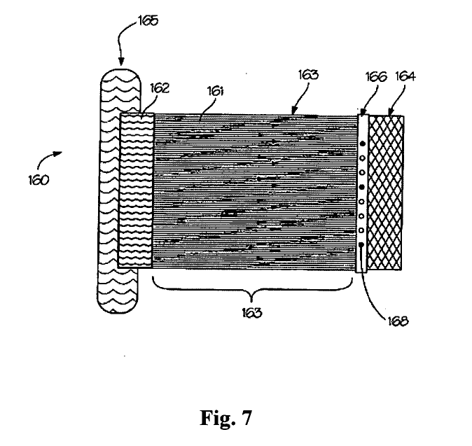

A yet further embodiment of the disclosed methods employs one or more conduits comprising a plurality of films. FIG. 7 depicts the use of capillary-channeled films in a lateral flow assay 160. In this embodiment, a series of films 161 are aligned on an underlying film 163 in an ordered array. The first end 162 of the film/film composite defines a collection region. The first end is exposed to a solvent reservoir 165 and the fluid flows along the films to the second end 164. Between the first end and the second end a detector array 166 is defined. The collection region 162 is dipped into, or passed through, the sample-containing stream. Solvent reservoir 165 is provided for the case where the sample is pre-immobilized and must be transported down the film structure by a second mobile phase fluid. Different from what is shown in FIG. 6, eight chemically selective indicators 168 are present in adjacent alignment. In this illustration, of the eight species for which the fluid is being tested, three, those indicated by the first, fourth and bottom indicators, are present.

As it relates to identifying organelles and cellular material, for example, cellular debris, a conduit comprising one or more channels having one or more chemically selective recognition substrates are present along the length of a plurality of films in a spaced, rather than adjacent, configuration. Furthermore, embodiments in which only one chemically selective recognition substrate is present along the length of a plurality of films or at least one channeled film are useful in a variety of consumer end-use applications.

### Substrates

Further disclosed herein is a polymeric substrate comprising:
a) a fluid conduit having a first end and a second end disposed opposite the first end, the conduit having at least one polymer film disposed therein, the at least one polymer film defining a plurality of capillary channels capable of wicking a fluid;
b) a probing positioning at a location along the conduit between the first end and the second end of the conduit; and
c) an instrument aligned with the probing position.

The substrates comprise at least one fluid conduit having closed ends. In one embodiment the conduit comprises a plurality of polymer films deposed thereon wherein the polymer films define capillary channels capable of transporting a fluid comprising cells, organelles, and/or other cellular components including debris, by wicking action from a point wherein the fluid is added to the conduit, along the channels, and across a location along the conduit wherein an instrument is aligned with a selected probing position. At the probing position the instrument provides a measurement to the user. The measurement can be the number of cells present in the fluid or the type of cells in the fluid. In addition, as described herein above, one or more of the capillary channels can be modified to comprise a cellular recognition substrate for determining the cell type or the presence of a particular type of organelle.

As such, the conduits are comprised of the same polymeric materials described further herein. In addition, the polymers forming the films that comprise the capillaries can be modified as described herein. One method of providing a recognition substrate/element to the conduits is to use a micron/submicron printer.

The conduits can further comprise a reservoir for collecting the sample fluid once the fluid has passed the probing position. The reservoir serves to collect the fluid and to maintain the wicking of the sample fluid along the capillaries. In one embodiment, the substrate is aligned such that all of the capillaries have their fluids collected in a common or shared reservoir.

### Surface Recognition Substrates

The disclosed methods utilize fluid conduits comprising one or more polymer films deposed thereon, wherein the film defines a plurality of capillary channels. Because of the polymers that comprise the films, the capillary channels can wick a fluid from a first point to a second point desirable by the user.

As such, the capillary channels can further comprise one of more surface recognition substrates. The surface recognition substrates can be small molecules, peptides, proteins, DNA/RNA, antibodies, or other molecules with biological function or which can recognize a specific cellular surface receptor and thereby distinguish between one cellular species and others that are present in the test fluid. In one embodiment, the surface recognition substrate can also be a dye molecule or biological stain that can react with a certain category of organelle or cellular material. In a further embodiment, a specific tagging agent can be applied to cells prior to delivery to the conduit; in this case, the tagging agent is recognized by a surface recognition substrate.

As described herein above, the surface recognition substrate can be applied to a modified polymer surface either chemically, as in a covalent bond, or electrostatically, for example as a carboxylic acid/amine ion pair. The surface recognition substrates can also be used to modify the flow of cells, for example, to provide a funnel wherein cells are kept from bunching together, thereby allowing the detection device to be able to clearly count or sort cells or cell types. This embodiment is also useful for methods wherein the detection device is capable of visually recording the passage of cells through the detection field.

### General Procedure

Intact cells (living or fixed), purified organelles, or cellular debris labeled with a luminescent dye are suspended in buffer (i.e. phosphate buffered saline (PBS)) and added at the input end of the capillary-channeled polymer. The cells, organelles, or cellular debris migrate slowly along the capillary-channeled polymer film, pass through the detection window, and are finally eluted off the capillary-channeled polymer film into an absorbant pad or other absorbing material. Particles can be counted using standard commercial image processing software after which a histogram of the counts can be plotted.

### EXAMPLE 1

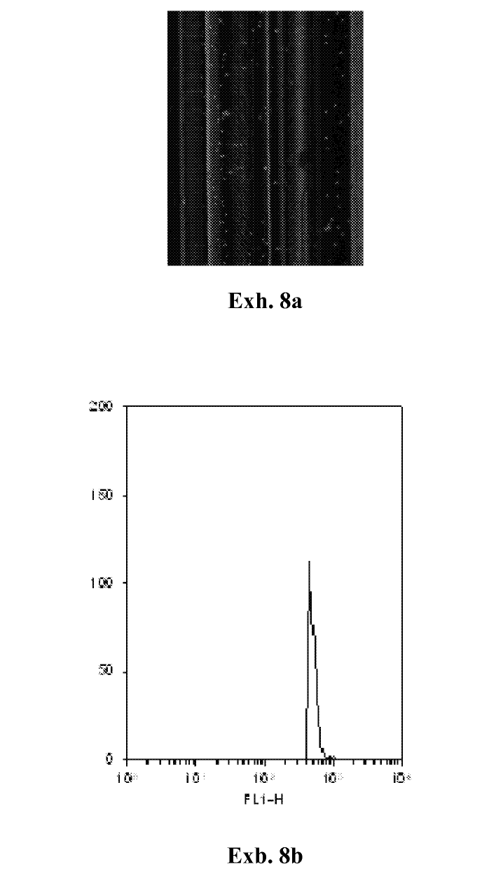

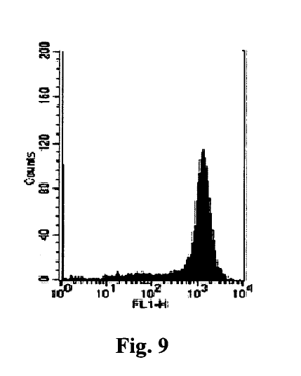

Intact TH-1 cells were labeled with 1-10 μM BCECF-AM by incubating the cells for 30-60 minutes at 37° C. The cells were re-suspended in phosphate buffered saline (PBS) and added to the input or reagent end of a horizontally positioned capillary-channeled polymer polyester film. The film was placed on an inverted research-grade fluorescence microscope and a time-lapse movie acquired using 485 nm excitation from a Xe arc lamp and 525 nm emission imaged onto a CCD camera (50-100 ms exposure times, 10× objective) while the labeled cells moved through the detection window. Fluorescent particles (labeled cells) were tracked and their fluorescence intensity measured using standard image processing software. FIG. 8A shows a single image of labeled TH-1 cells that are being transported within the channels by way of the wicking action and FIG. 8B depicts a histogram of number of cells vs. intensity obtained from a time-lapse movie taken of the cells as they passed the probing position. As a control, BCECF-AM labeled TH-1 cells were also run through a standard flow cytometer and the data were compared. FIG. 9 shows the flow Cytometry histogram using a Becton-Dickenson FACScan flow cytometer (488 nm excitation, 535 nm fluorescence emission). A comparable distribution of cells was observed in the capillary-channeled polymer film flow cytometry as compared to data from the commercially available flow cytometer with the exception of different intensity scales due to the use of different types of detectors.

### EXAMPLE 2

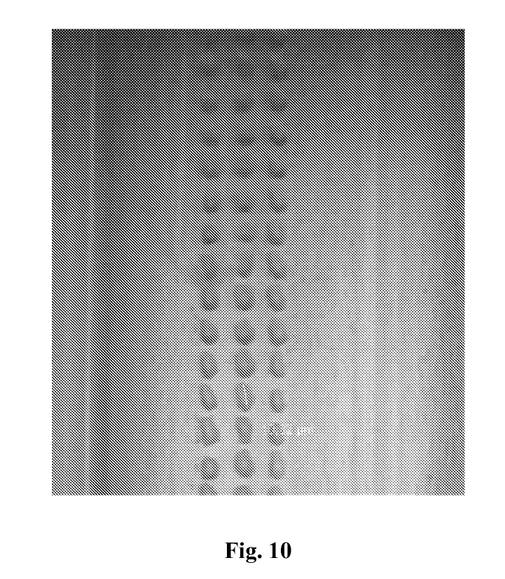

As disclosed herein, single capillary channels or the capillary-channeled polymer films can be derivatized. Derivatization of the polymer films allows for multiple in parallel analysis, for example, counting and sorting of cells. The recognition substrates/elements can be delivered to the conduits using an open-channeled modified atomic-force microscope tip such as the NANOENABLER® nanoprinter disclosed in U.S. Patent Application Publication 20050266149. FIG. 10 is a transmitted light (bright field) image of a capillary-channeled film where a proteinaceous recognition substrate/element diluted in phosphate buffer+50% glycerol was printed within a single channel. FIG. 10 shows an array of spots, vectors or lines can also be printed using this method along the length on the channel.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A method for counting cells, comprising:
a) providing a fluid conduit having a first end and a second end disposed opposite the first end, the conduit having at least one polymer film disposed therein, the at least one polymer film defining a plurality of capillary channels capable of wicking a fluid;
b) defining a probing positioning at a location along the conduit between the first end and the second end of the conduit;
c) aligning an instrument with the probing position, the instrument being configured for detecting and/or counting cells;
d) moving fluid containing the cells through the capillaries; and
e) detecting the number of cells with the instrument.

2. The method according to claim 1, wherein the fluid is delivered to the fluid conduit by a pump.

3. The method according to claim 1, wherein the fluid conduit comprises a plurality of co-linear capillary channels.

4. The method according to claim 1, wherein step (e) is achieved by a first detection device that utilizes a detection method chosen from visual detection, phase contrast, differential interference contrast, or bright field imaging, IR absorbance, UV-visible absorbance, fluorescence, Raman spectroscopy, or mass spectrometry.

5. The method according to claim 1, wherein the fluid conduit comprises a polymer chosen from poly(ethylene terephthalate), nylon, or polypropylene.

6. The method according to claim 1, wherein the fluid conduit comprises a surface modified polymer chosen from poly(ethylene terephthalate), nylon, or polypropylene.

7. The method according to claim 1, wherein the fluid conduit surface further comprises a means for selectively interacting with one or more types of cells.

8. The method according to claim 1, wherein the fluid conduit surface further comprises a cellular surface recognition substrate.

9. The method according to claim 8, wherein the cellular surface recognition substrate modifies the rate of flow of one or more cell types.

10. The method according to claim 8, wherein the cellular surface recognition substrate is a second detection method.

11. The method according to claim 10, wherein the first and second detection devices each report a different cellular recognition parameter.

12. The method according to claim 1, wherein the fluid conduit further comprises a fluid sink for collecting the fluid.

13. A method for determining the presence of cellular organelles, comprising:
a) providing a fluid conduit having a first end and a second end disposed opposite the first end, the conduit having at least one polymer film disposed therein, the at least one polymer film defining a plurality of capillary channels capable of wicking a fluid;
b) defining a probing positioning at a location along the conduit between the first end and the second end of the conduit;
c) aligning an instrument with the probing position, the instrument being configured for detecting and/or counting cells;
d) moving fluid containing cellular organelles through the capillaries; and
e) detecting the number of cells with the instrument.

14. The method according to claim 13, wherein the fluid is delivered to the fluid conduit by a pump.

15. The method according to claim 13, wherein the fluid conduit comprises a plurality of co-linear capillary channels.

16. The method according to claim 13, wherein step (e) is achieved by a first detection device that utilizes a detection method chosen from visual detection, phase contrast, differential interference contrast, or bright field imaging, IR absorbance, UV-visible absorbance, fluorescence, Raman spectroscopy, or mass spectrometry.

17. The method according to claim 13, wherein the fluid conduit comprises a polymer chosen from poly(ethylene terephthalate), nylon, or polypropylene.

18. The method according to claim 13, wherein the fluid conduit comprises a surface modified polymer chosen from poly(ethylene terephthalate), nylon, or polypropylene.

19. The method according to claim 13, wherein the fluid conduit surface further comprises a means for in selectively interacting with one or more types of organelles.

20. The method according to claim 13, wherein the fluid conduit surface further comprises a cellular organelle recognition substrate.

21. The method according to claim 20, wherein the cellular organelle recognition substrate interacts with one or more organelles.

22. The method according to claim 20, wherein the cellular organelle recognition substrate is a second detection method.

23. The method according to claim 22, wherein the first and second detection devices each report a different cellular recognition parameter.

24. The method according to claim 13, wherein the fluid conduit further comprises a fluid sink for collecting the fluid.

25. A method for determining the presence of cellular material, comprising:
a) providing a fluid conduit having a first end and a second end disposed opposite the first end, the conduit having at least one polymer film disposed therein, the at least one polymer film defining a plurality of capillary channels capable of wicking a fluid;
b) defining a probing positioning g at a location along the conduit between the first end and the second end of the conduit;
c) aligning an instrument with the probing position, the instrument being configured for detecting and/or counting cells;
d) moving fluid containing cellular material through the capillaries; and
e) detecting one or more cellular materials with the instrument.

26. The method according to claim 25, wherein the fluid is delivered to the fluid conduit by a pump.

27. The method according to claim 25, wherein the fluid conduit comprises a plurality of co-linear capillary channels.

28. The method according to claim 25, wherein step (e) is achieved by a first detection device that utilizes a detection method chosen from visual detection, phase contrast, differential interference contrast, or bright field imaging, IR absorbance, UV-visible absorbance, fluorescence, Raman spectroscopy, or mass spectrometry.

29. The method according to claim 25, wherein the fluid conduit comprises a polymer chosen from poly(ethylene terephthalate), nylon, or polypropylene.

30. The method according to claim 25, wherein the fluid conduit comprises a surface modified polymer chosen from poly(ethylene terephthalate), nylon, or polypropylene.

31. The method according to claim 25, wherein the fluid conduit surface further comprises a means for in selectively interacting with one or more types of cellular material.

32. The method according to claim 52, wherein the fluid conduit surface further comprises a cellular material recognition substrate.

33. The method according to claim 32, wherein the cellular material recognition substrate interacts with one or more species of cellular material.

34. The method according to claim 32, wherein the cellular material recognition substrate is a second detection method.

35. The method according to claim 34, wherein the first and second detection devices each report a different cellular species recognition parameter.

36. The method according to claim 13, wherein the fluid conduit further comprises a fluid sink for collecting the fluid.

37. A polymeric substrate comprising:
d) a fluid conduit having a first end and a second end disposed opposite the first end, the conduit having at least one polymer film disposed therein, the at least one polymer film defining a plurality of capillary channels capable of wicking a fluid;
e) a probing positioning at a location along the conduit between the first end and the second end of the conduit; and
f) an instrument aligned with the probing position.

38. The substrate according to claim 37, wherein the instrument is a detection device chosen from visual detection, phase contrast, differential interference contrast, or bright field imaging, IR absorbance, UV-visible absorbance, fluorescence, Raman spectroscopy, or mass spectrometry.

39. The substrate according to claim 37, wherein the at least one polymer film is extrudable at a temperature of from about 150° C. to about 350° C.

40. The substrate according to claim 37, wherein the fluid conduit comprises at least one polymer film chosen from poly(ethylene terephthalate), nylon, or polypropylene.

41. The substrate according to claim 37, wherein the fluid conduit comprises a surface modified polymer film chosen from poly(ethylene terephthalate), nylon, or polypropylene.

42. The substrate according to claim 37, wherein the fluid conduit surface further comprises a means for selectively interacting with one or more types of cells.

43. The substrate according to claim 42, wherein the fluid conduit surface further comprises a cellular surface recognition substrate.

44. The substrate according to claim 42, wherein the cellular surface recognition substrate modifies the rate of flow of one or more cell types.

45. The substrate according to claim 42, wherein the cellular surface recognition substrate is a second detection method.

46. The substrate according to claim 45, wherein the first and second detection devices each report a different cellular recognition parameter.

47. The substrate according to claim 37, wherein the fluid conduit further comprises a fluid sink for collecting the fluid.

48. The substrate according to claim 37, further comprising a pump for delivering a sample to the conduit.
